(19) United States
(12) Patent Application Publication
(10) Pub. No.: US 2024/0277432 A1
(43) Pub. Date: Aug. 22, 2024

(54) METHOD OF TELEOPERATION PREPARATION IN A TELEOPERATED ROBOTIC SURGERY SYSTEM AND RELATED SYSTEM

(71) Applicant: MEDICAL MICROINSTRUMENTS, INC., Wilmington, DE (US)

(72) Inventors: Matteo TANZINI, Pisa (IT); Michael John PROCTOR, Pisa (IT); Giuseppe Maria PRISCO, Pisa (IT); Massimiliano SIMI, Pisa (IT)

(21) Appl. No.: 18/570,447

(22) PCT Filed: Jun. 16, 2022
(86) PCT No.: PCT/IB2022/055572

§ 371 Date: Dec. 14, 2023

(30) Foreign Application Priority Data
Jun. 17, 2021 (IT) 102021000015902

(51) Int. Cl. A61B 34/35; A61B 34/00; A61B 34/30

(52) U.S. Cl. CPC A61B 34/35; A61B 2034/305; A61B 2034/715

(57) ABSTRACT

A method of teleoperation preparation in a teleoperated robotic surgery system is performed during a non-operating step. The applicable robotic system includes motorized actuators, and a surgical instrument. The surgical instrument includes an articulated end-effector having a degree/degrees of freedom. The surgical instrument includes a pair of antagonistic tendons, mounted in the surgical instrument operatively connected/connectable to the motorized actuators and to the respective end-effector links. The antagonistic tendons actuate degree/degrees of freedom associated therewith, between the degree/degrees of freedom thus determining antagonistic effects. The method includes establishing a univocal correlation motorized actuators' movements and articulated end-effector movements. A holding step includes tensile-stressing a pair of antagonistic tendons, and holding the tendons in a tensile-stressed state, by applying a holding force to the tendons, adapted to determine a loaded state of the tendons. A command is given to enter teleoperation. A corresponding teleoperated robotic surgery system performs the method.

### TECHNOLOGICAL BACKGROUND OF THE INVENTION

### Field of Application

The present invention relates to a method of teleoperation preparation in a teleoperated robotic surgery system and to the related robotic system.

Therefore, the present description more generally relates to the technical field of operational control of robotic systems for teleoperated surgery.

### Description of the Prior Art

In a teleoperated robotic surgery system, the actuation of one or more degrees of freedom of a slave surgical instrument is generally enslaved to one or more master control devices configured to receive a command imparted by the surgeon. Such a master-slave control architecture typically comprises a control unit which can be housed in the robotic surgery robot.

Known hinged/articulated surgical instruments for robotic surgery systems include actuation tendons or cables for transmitting motion from the actuators, operatively connected to a backend portion (actuation interface) of the surgical instrument, distally to the tips of the surgical instrument intended to operate on a patient anatomy and/or to handle a surgical needle, as for example shown in documents WO-2017-064301 and WO-2018-189729 in the name of the same Applicant. Such documents disclose solutions in which a pair of antagonistic tendons is configured to implement the same degree of freedom as the surgical instrument. For example, a rotational joint of the surgical instrument (degree of freedom of pitch and degree of freedom of yaw) is controlled by applying tensile force applied by the pair of the aforesaid antagonistic tendons.

For example, document WO-2014-070980 shows a surgical instrument having a backend portion having a winch around which are wound, in opposite directions, both two antagonistic movement tendons of a degree of freedom of the surgical instrument. A preload spring exerts an elastic action of influence to keep the tendons taut.

Further known are surgical instruments in which the same pair of tendons is capable of simultaneously actuating more than one degree of freedom, such as shown in WO-2010-009221 in which only two pairs of tendons are configured to control three degrees of freedom of the surgical instrument.

Typically, tendons for robotic surgery are made in the form of metal cords (or strands) and are wound around pulleys mounted along the surgical instrument. Each tendon can be mounted on the already elastically preloaded instrument, i.e., pre-conditioned prior to assembly on the instrument, so that each tendon is always in a tensile state in order to provide a rapid actuation response of the degree of freedom of the surgical instrument when activated by the actuators and, consequently, to provide good control over the degree of freedom of the surgical instrument.

In general terms, all the cords are subject to elongation when subjected to loads. New cords of the intertwined type typically have a high elongation of plastic-elastic nature when under load due at least in part to the unraveling of the fibers forming the cord.

For this reason, before assembly on the surgical instrument, it is common practice to subject the new tendons to a high initial load in order to remove the residual plasticity of the drawing and intertwining process or of the material itself.

In general, the cords typically have three elongation elements:
(1) elastic elongation deformation, which is recovered when the tensile load stops;
(2) recoverable deformation, i.e., a relatively small deformation which is gradually recovered over a certain period of time and is often a function of the nature of the intertwinement, and can take a period of time between a few hours and a few days when not subjected to any load;
(3) non-recoverable permanent elongation deformation.

The permanent elongation deformation, as described above, can be achieved by a cord breaking-in procedure, performed prior to assembly on the instrument, which can comprise loading and unloading cycles and involve a plastic elongation deformation of the fibers themselves.

Viscous creep deformation under tensile load is a time-dependent effect which affects some types of intertwined cords when subject to fatigue and can be recoverable or non-recoverable typically depending on the intensity of the applied load.

Generally, the fatigue behavior of polymer fibers differs from the fatigue behavior of metal fibers in that the polymer fibers are not subject to crack propagation breakage, as instead are metal fibers, although cyclic stresses may lead to other forms of breakage.

WO-2017-064306 in the name of the same Applicant shows a solution of an extremely miniaturized surgical instrument for robotic surgery, which uses tendons adapted to support high radii of curvature and at the same time adapted to slide on the surfaces of the rigid elements, commonly referred to as “links”, which form the hinged/articulated tip of the surgical instrument. In order to allow such a sliding of the tendons, the tendons-link sliding friction coefficient must be kept as low as possible, and the above-mentioned document teaches to use tendons formed by polymer fibers (rather than using steel tendons).

Although advantageous from many points of view, and indeed as a consequence of the fact that an extreme miniaturization of the surgical instrument is obtained by virtue of the use of the aforesaid tendons formed by polymeric fibers, in the context of this solution it becomes even more important to avoid the occurrence of an elongation or a shortening (contraction) of the tendons under operating conditions of the surgical instrument, because with the same variation in length, as the size decreases, the uncontrollability effects of the miniaturized surgical instrument would be accentuated.

Metal tendons have a modest recoverable elongation and the aforementioned preloading processes performed before assembly on the surgical instrument are typically sufficient to completely remove the residual plasticity, while the preload to which they are subject when assembled maintains an immediate reactivity in use.

For example, document US-2018-0228563 shows a strategy which includes, in preparation for a teleoperation, placing two antagonistic tendons in a tensile state, independently, and then mechanically coupling the actuators of the two antagonistic tendons, to obtain the tendons taut so as to provide a rapid response when stressed under operating conditions.

Otherwise, the tendons made of polymer materials have high elongations due to the contributions described above; moreover, the preloading processes, if carried out before assembly, do not prevent the tendon from quickly recovering a large fraction of the recoverable elongation as soon as the tendons are subject to low tensile loads. If on the one hand the forecasting of any high assembly preloads prevents the recovery of the deformation, on the other hand it aggravates the creep process of the polymer tendon even when not in use, forcing the tendon to stretch almost indefinitely and weaken, and therefore is not a viable strategy.

For example, intertwined cords formed by high molecular weight polyethylene fibers (HMWPE, UHMWPE) are usually subject to non-recoverable deformation, while intertwined cords of aramid, polyesters, liquid crystal polymers (LCP), PBO (Zylon®), nylon are less affected by this feature.

In the case of surgical instruments, the variation in the length of the tendons attributable to the tendon elongation phenomenon described above, as well as the recovery of the elongation, is highly undesirable, in particular when under operating conditions, because it would necessarily impose objective complications in the control in order to maintain an adequate level of precision and accuracy of the surgical instrument itself.

As a further example of the background art, U.S. patent application US-2020-0054403 can be cited, which shows an engagement procedure of a surgical instrument at an actuation interface of a robotic system, in which motorized rotary disks of the robotic system engage with corresponding rotary disks of the surgical instrument in turn connected to actuation cables of degrees of freedom of the end-effector of the surgical instrument. The engagement procedure described therein allows recognizing whether the surgical instrument is operatively engaged with the robotic system, evaluating the response perceived by the motorized rotary disks of the robotic system.

Therefore in brief, the need is felt to avoid or at least minimize the lengthening or recovery of the actuation tendon of one or more degrees of freedom of the surgical instrument during use or over time, as well as to avoid, or at least minimize, the lost motion deriving from an undesirable lengthening or recovery of the tendon in operating conditions, such as during a teleoperation or entering a new teleoperation state after a period of non-teleoperation (non-solicitation), without for this reason imposing an increase in the dimensions of the surgical instrument, particularly of the distal hinged/articulated portion thereof.

Meanwhile, the need is felt to provide a solution which, although simple, is capable of ensuring a high level of controllability of the surgical instrument, and is thus reliable when in operating conditions such as during a teleoperation, and meanwhile does not hinder a boosted miniaturization of the surgical instrument, especially in the distal hinged/articulated portion thereof.

### SUMMARY OF THE INVENTION

It is the object of the present invention to provide a method of teleoperation preparation in a teleoperated robotic surgery system, which allows overcoming at least partially the drawbacks complained above with reference to the background art, and to respond to the aforementioned needs particularly felt in the technical field considered. Such an object is achieved by a method according to claim 1.

Further embodiments of such a method are defined by claims 2-29.

It is further the object of the present invention to provide a teleoperated robotic surgery system capable of performing and/or adapted to be controlled by the aforesaid method. Such an object is achieved by a system according to claim 30.

Further embodiments of such a system are defined by claims 31-49.

More in particular, it is an object of the present invention to provide a solution in line with the aforesaid technical requirements, with the features summarized below.

Possible procedures for preparing for teleoperation, in general terms, are exemplified in FIG. 10, in which steps of engaging the surgical instrument, conditioning (also referred to as the “pre-conditioning” step), and alternating holding and teleoperating steps are mentioned. The present disclosure focuses on the latter in particular. For example, the steps of engagement, conditioning and holding can be states in which the robotic system works autonomously, i.e., non-teleoperated.

In fact, by virtue of the suggested solutions, it is possible to carry out a teleoperation preparation step comprising a teleoperation preparation holding procedure.

In the following, reference will be made to a “holding procedure” of a preparation step also with the terminology (equivalent for the purposes of this disclosure) “holding step”.

The aforesaid teleoperation preparation step, comprising a holding procedure, is preferably performed before each teleoperating step in which at least one surgical instrument of a slave device fully follows (i.e., fully enslaved tracking) at least one master device.

The holding procedure can be performed after an initialization step comprising an initial engagement procedure in which the surgical instrument is engaged to the slave robotic platform, and before a teleoperating step.

The holding procedure can be performed after an initialization step comprising an initial conditioning step, in which the surgical instrument is subject to a conditioning of the tendons thereof (also referred to as “pre-stretching”), and before a teleoperating step.

The holding procedure can be performed between two adjacent teleoperating steps, such as between the end of one teleoperating step and the beginning of the next teleoperating step.

For example, between two adjacent teleoperating steps an intermediate step can be interposed in which the surgical instrument of the slave device does not follow the master device, such as a suspended teleoperating step and/or a limited teleoperating step and/or an accommodation step and/or a rest step.

In light of the above, a first holding step can be performed after the initialization step, comprising a conditioning step, and before a first teleoperating step; further, a second holding step can be performed between the aforesaid first teleoperating step and a second teleoperating step following the first teleoperating step. Therefore, those skilled in the art will appreciate that further holding steps can be performed at the end of each teleoperating step and before a subsequent contiguous teleoperating step. The number of successive and contiguous teleoperating steps which can be performed during a teleoperated robotic surgery operation can depend on various contingent and specific needs.

In other words, after the initialization step, comprising the engagement procedure, and the conditioning procedure, one or more cycles are performed comprising a holding step and a teleoperating step following the holding step.

Performing the at least one holding step allows the tendons of the surgical instrument to be kept in a tensile stressed state upon entry into a teleoperating step, ensuring a swift response of the tendons.

For example, at the end of the initialization step comprising the aforesaid conditioning procedure, the execution of the holding step allows avoiding the relaxation of the tendons in view of the teleoperating step, holding the conditioning level of the tendons reached during the conditioning step (“pre-stretch”).

By virtue of the holding step, it is possible to rebalance the reference position of the actuators of the slave robotic system and/or of the transmission elements of the surgical instrument at the end of a teleoperating step during which the tendons of the surgical instrument can have varied the length thereof, for example due to sliding friction and/or recovery of the recoverable deformation.

In fact, during a teleoperating step in which the surgical instrument completely follows the master device, it can occur that the performance of at least some tendons undergoes degradation due to intensive actuation of the degrees of freedom of the surgical instrument, an actuation which can require the tendons to describe high radii of curvature (such as with reference to degrees of freedom of pitch/yaw).

Alternatively or in addition, a prolonged and relatively high tensile level of a subset of tendons (e.g., of one or two pairs of antagonistic tendons to hold a prolonged gripping condition or “grip” of the tips of the surgical instrument on a surgical needle and/or on a biological tissue) can occur during a teleoperating step. This can generate, in addition to the degradation of the performance of the two antagonistic tendons connected to the grip degree of freedom, also a kinematic imbalance due to the fact that a subset of the total number of tendons are subject to a more intense actuation.

Performance degradation can increase as the duration of the teleoperating step increases as well as the duration of the prolonged gripping condition increases.

The application of relatively high tensile forces on the tendons can have the undesirable effect of causing a flattening of the transverse section of the tendons, and this can result in an increase in the contact surface of the tendon on the sliding surface thereof (e.g., a surface of a link of the hinged surgical instrument), which in turn results in an increase in the friction forces, contributing even more markedly to the degradation of the tendon performance.

The holding step preferably ends with the application of relatively low forces in order to avoid this flattening/crushing of the tendons before entering the teleoperating step.

Otherwise, during the holding step, the surgical instrument preferably does not follow the master device, and therefore the surgical instrument can be held in stationary conditions according to a kinematic point of view.

By virtue of the suggested solutions, it is possible to eliminate or at least minimize the recoverable elongation from the at least one tendon to actuate a degree of freedom of the surgical instrument and it is possible to obtain a precise transfer of the actuation action applied on the tendon even if the teleoperation is interrupted and/or suspended.

By virtue of the suggested solutions, it is possible to eliminate or at least minimize the recovery of the recoverable elongation of the at least one tendon to actuate a degree of freedom of the surgical instrument and it is possible to obtain a precise and stable transfer of the actuation action during the subsequent teleoperating step even if the teleoperation has previously been interrupted and/or suspended.

By virtue of the suggested solutions, an improved accuracy of the kinematic correspondence between master and slave is provided during a teleoperation as well as during two adjacent teleoperating steps.

By virtue of the suggested solutions, lost motion due to an undesirable lengthening of the tendon when in operating conditions is avoided or at least reduced to a minimum.

By virtue of the suggested solutions, a satisfactory stabilization of the physical features of the surgical instrument is provided.

By virtue of the suggested solutions, improved control over the degrees of freedom of the surgical instrument is provided.

### BRIEF DESCRIPTION OF THE DRAWINGS

Further features and advantages of the method according to the invention will become apparent from the following description of preferred exemplary embodiments, given by way of non-limiting indication, with reference to the accompanying drawings, in which:

FIG. 1 shows in axonometric view a robotic system for teleoperated surgery, according to an embodiment;

FIG. 2 shows in axonometric view a portion of the robotic system for teleoperated surgery of FIG. 1;

FIG. 3 shows in axonometric view a distal portion of a robotic manipulator, according to an embodiment;

FIG. 4 shows in axonometric view a surgical instrument, according to an embodiment, in which tendons are schematically diagrammatically shown in a dashed line;

FIG. 5 diagrammatically shows in plan and partially sectioned view for clarity the actuation of a degree of freedom of an articulated end-effector device (or end-effector) of a surgical instrument, according to a possible operating mode;

FIG. 6 is a diagram which takes the example shown in FIG. 5 showing a possible conditioning step of a method of teleoperation preparation, according to a possible operating mode;

FIG. 7 diagrammatically shows the actuation of a degree of freedom of an articulated end-effector device of a surgical instrument, according to a possible operating mode;

FIGS. 8A, 8B, 8C and 8D show respective graphs showing the time trends of force application to motorized actuators, according to various sequences of steps, as a function of time, according to an operating mode;

FIG. 9 is a diagrammatic sectional view of a portion of a surgical instrument and a portion of a robotic manipulator showing the actuation of a degree of freedom of a surgical instrument, according to a possible operating mode;

FIG. 10 is a flow diagram showing steps of a method comprising preparation for teleoperation and teleoperation, according to a possible operating mode;

FIGS. 11 and 12 are two flow diagrams showing steps of a method comprising preparation for teleoperation and teleoperation, according to two respective possible operating modes;

FIG. 13 shows in axonometric view an end device of the surgical instrument, according to an embodiment of the present invention, and a gripping action performed by two pairs of antagonistic tendons, according to a possible operating mode.

### DETAILED DESCRIPTION

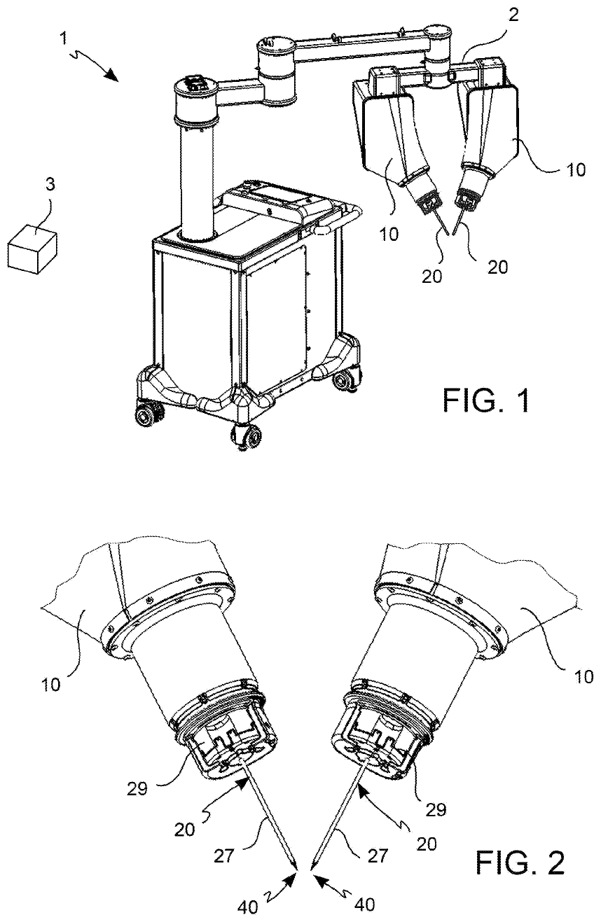

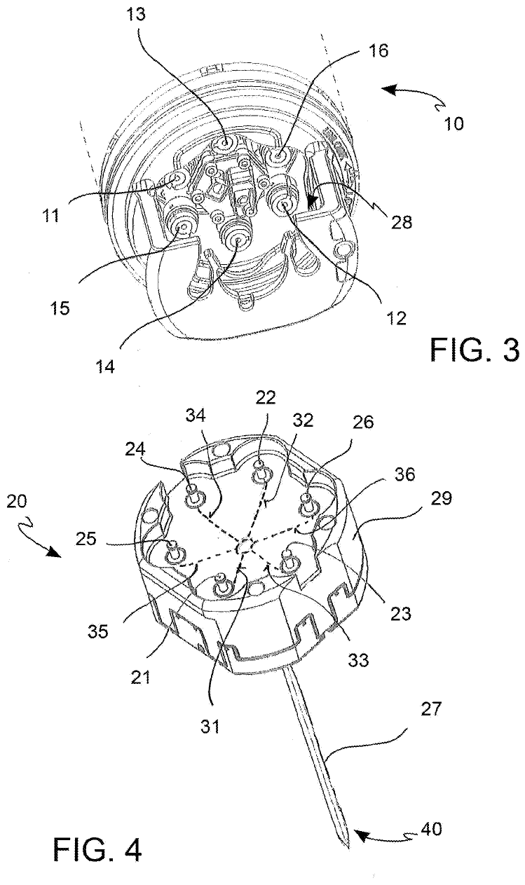

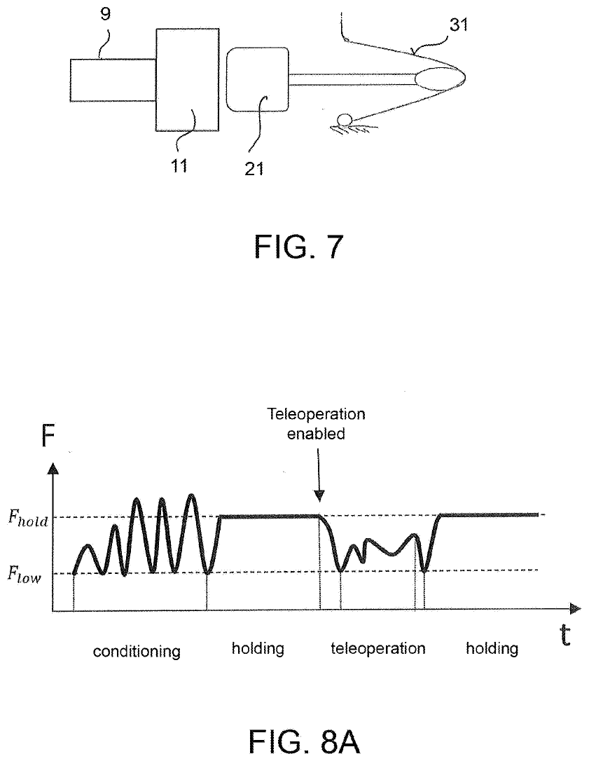

With reference to FIGS. 1-13, a method of teleoperation preparation in a surgical teleoperated robotic system 1 is described, to be performed during a non-operating step, in which the system is not performing a teleoperation.

The aforesaid robotic system 1, to which the method is applicable, comprises a plurality of motorized actuators 11, 12, 13, 14, 15, 16, and at least one surgical instrument 20.

The surgical instrument 20 further comprises an articulated end-effector device 40 (i.e., articulating tip 40) having at least one degree of freedom (P, Y, G). The articulated end-effector device 40 is also commonly referred to as the “articulated terminal device” or “articulated end-effector” or “hinged end-effector” (such definitions will be used hereinafter as synonyms).

The surgical instrument 20 further comprises at least one pair of antagonistic tendons (31, 32), (33, 34), (35, 36), mounted in the aforesaid surgical instrument 20 so as to be operatively connected or connectable to both the motorized actuators and to the respective links (or rigid connection elements) of the end-effector device 40. The tendons of the aforesaid pair of antagonistic tendons are configured to actuate/implement at least one degree of freedom associated therewith, among the aforesaid at least one degree of freedom P, Y, G, thus determining antagonistic effects.

The method comprises the following steps:
(i) establishing a univocal correlation between a set of movements of the motorized actuators 11, 12, 13, 14, 15, 16 of the robotic system 1 and a respective movement of the articulated end-effector device 40 of the surgical instrument 20;
(ii) performing a holding step in turn comprising:
   stressing, through tensile-stressing, at least one pair of antagonistic tendons (31, 32), (33, 34), (35, 36) and keeping such tendons in a tensile-stressed state, by applying a holding force Fhold to the tendons (for example, by means of a feedback control loop) which is adapted to determine a loaded state of the tendons: the aforesaid stressing step is determined by the motorized actuators;
   providing a command indicating a will to enter teleoperation;
   enabling the surgical instrument 20 to enter a teleoperation state.

In accordance with an embodiment, the method is applied to a surgical instrument further comprising a plurality of transmission elements 21, 22, 23, 24, 25, 26, each operatively connectable with a respective at least one motorized actuator 11, 12, 13, 14, 15, 16.

In such a case, the aforesaid stressing step is performed by the transmission elements 21, 22, 23, 24, 25, 26, operated and controlled by the respective motorized actuators.

In other words, the transmission system of the surgical instrument 20 for transmission of the action imparted by the motorized actuators comprises said tendons and preferably also said transmission elements which interface with respective motorized actuators of the robotic manipulator.

The transmission elements are preferably rigid elements. Thereby the action of a motorized actuator is transmitted to the respective tendon without attenuations/distortions which could instead be introduced if the transmission element was an elastic and/or damping element, for example.

According to an implementation option, the operating connection between the tendons of the surgical instrument and the respective motorized actuators can be a releasable connection.

In accordance with an implementation option, the operating connection between the tendons of the surgical instrument and the respective motorized actuators can be a direct or indirect connection, for example by interposing respective transmission elements, which can be connected to the tendons.

In accordance with an embodiment. the method comprises, after steps (i)-(ii), the step (iii) of teleoperating by means of the surgical instrument 20 of the robotic system (1).

According to an implementation option of the method, the holding (ii) and teleoperating (iii) steps are repeated, so that a holding step (ii) is performed between two adjacent teleoperating steps (iii).

In accordance with an embodiment of the method, in which a kinematic zero position of each of the motorized actuators 11, 12, 13, 14, 15, 16 is defined, the method comprises, during the holding step (ii) and after the aforesaid step of stressing at least one pair of antagonistic tendons, the further step of storing a possible position offset of each of the motorized actuators 11, 12, 13, 14, 15, 16 with respect to the respective stored kinematic zero position.

According to an embodiment of the method, during the holding step (ii), the step of stressing at least one pair of antagonistic tendons comprises at least one loading and unloading cycle, in which each loading and unloading cycle includes applying a high force Fhold to determine a loaded state of the pair of tendons and applying a low force Flow to determine an unloaded state of the pair of tendons.

In such a case, such a high force corresponds to said holding force Fhold, and such a low force Flow is a lower force than said holding force Fhold.

According to an implementation option, in each of the aforesaid loading and unloading cycles, first the low force Flow is applied and then the high or holding force Fhold is applied.

In accordance with an implementation option, during the holding step (ii), between the step of providing a command indicating the will to enter teleoperation and the step of enabling entry into a teleoperation state, the further step of applying the aforesaid low force Flow to the tendons is provided, so as to tensile-stress the tendons according to the aforesaid unloaded state of the loading and unloading cycle.

According to an embodiment, the method comprises the further steps of detecting the forces applied to all the tendons upon exiting a teleoperating step, identifying the minimum force Fmin among the detected forces, and then bringing all the tendons to an intermediate stress condition corresponding to said minimum force value Fmin.

It should be noted that such a force Fmin (so called because it is the lower force among all the forces detected at the exit from the teleoperation) corresponds to an intermediate stress condition, which then brings all the tendons to an intermediate force value Fmin between the values of the low and high forces: Flow<Fmin<Fhold.

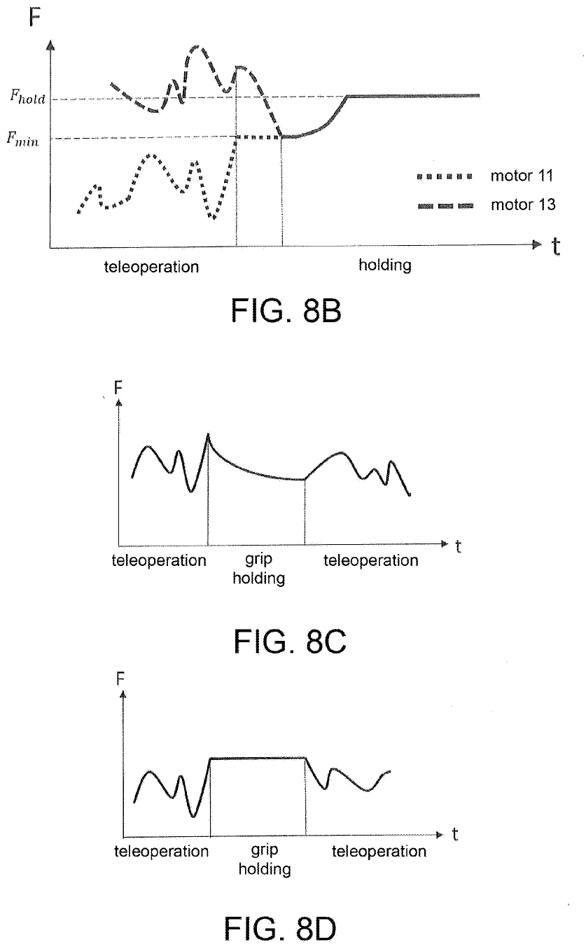

As already noted, and as also illustrated for example in FIG. 8B, the intermediate stress force Fmin is recorded exiting teleoperation.

Therefore, keeping the holding forces “low” and equal to each other, the drawback of inadvertently moving the degrees of freedom of “pitch”, “yaw” and “grip” of the articulating end-effector 40 during the holding step is avoided.

According to possible implementation options, the method further comprises, preferably, a subsequent step of bringing all the tendons to an unloaded stress condition, corresponding to the low force Flow; and/or a subsequent step of bringing all the tendons to a loaded stress condition, corresponding to the high holding force Fhold.

In accordance with an implementation option, the aforesaid step of bringing all the tendons to an intermediate stress condition corresponding to the minimum force value Fmin is performed following specific and/or different loading and/or unloading curves for each tendon, as a function of the starting force value detected for each tendon.

In accordance with an implementation option, the aforesaid step of applying the holding force Fhold to the tendons comprises:
bringing all the tendons to an intermediate stress condition corresponding to the aforesaid minimum force value Fmin, each tendon according to a respective specific load curve dependent on the respective detected starting force value, so that the load is equally distributed between the antagonistic tendons of one or more pairs of antagonistic tendons;
then bringing all the tendons to a load stress condition, corresponding to the aforesaid holding force Fhold.

In accordance with an embodiment of the method, the teleoperating step begins with a predeterminable teleoperating start force Fwork applied to the tendons which is lower than the aforesaid high holding force value Fhold.

According to an implementation option, the aforesaid predeterminable teleoperating start force Fwork is substantially equal to the low holding force Flow, i.e., Fwork=Flow.

According to an implementation option, the transition between the high holding force Fhold and the teleoperating start force Fwork is preferably controlled by the user by activating a control pedal.

Such a sequence of stresses is shown in FIG. 8A, in which it can be observed that, after each holding step, the force is lowered to the level Flow for the teleoperation start. The descent front from the high force Fhold to the low force Flow, to start the teleoperation, is controlled by the control pedal activated by the user, so that the entry into teleoperation is always intentional. The exit from teleoperation can instead be either intentional, by the user by means of pedal activation, or independently controlled by the robot, for example following an anomaly detected by a check.

It should also be noted that, in this embodiment, the low and high forces determine the following effects, from the point of view of tendon behavior when subject to various tensile states:
the low force Flow is ideally the minimum contact force which can be recorded between the motorized actuator 11 and the respective tendon 31 (or between the motorized actuator and the respective transmission element 21), which thus senses the contact; however, the low force Flow is such as not to determine the actuation of the degrees of freedom of the end-effector device;
the high force Fhold is the holding force which is provided and held to avoid relaxation i.e., recovery of the tendon deformation state.

In accordance with an embodiment of the method, the aforesaid step of stressing the tendons comprises measuring or detecting the force acting on the tendons during the loading cycle, and reaching the holding force value Fhold, by the motorized actuators, through a feedback force control procedure based on the actual force acting on the tendons as detected or measured.

For example, the effective force acting on the tendons is detected or measured by force sensors 17, 18 placed at the distal interface of the motorized actuators, so as to detect the contact force between motorized actuators and transmission elements, where provided.

According to an embodiment of the method, the aforesaid step of stressing the tendons comprises measuring or detecting the force acting on the tendons during the unloading cycle, and reaching the low force value Flow, by the motorized actuators, through a feedback force control procedure based on the actual force acting on the tendons as detected or measured.

In accordance with another embodiment of the method, the aforesaid step of stressing the tendons comprises measuring or detecting a position offset of the transmission elements 21, 22, 23, 24, 25, 26 or of the motorized actuators 11, 12, 13, 14, 15, 16 with respect to the respective initial values, predefined or stored at the end of the previous teleoperating step, and performing the loading cycle, by the motorized actuators, through a feedback position control procedure based on the aforesaid position offsets as detected or measured or stored.

According to an embodiment of the method, the aforesaid step of stressing the tendons comprises measuring or detecting a position offset of the transmission elements 21, 22, 23, 24, 25, 26, or of the motorized actuators 11, 12, 13, 14, 15, 16, with respect to the respective initial values, predefined or stored at the end of the previous teleoperating step, and performing the unloading cycle, by the motorized actuators, through a feedback position control procedure based on the aforesaid position offsets as detected or measured or stored.

In accordance with an embodiment of the method, during the holding step (ii), the at least one pair of tendons is stressed by means of a loaded state corresponding to a gripping action of the end-effector device 40 of the surgical instrument 20, so that during the holding step the surgical instrument is in a gripping condition.

This embodiment (which can be defined as “hold squeeze”, i.e., grip holding) is preferably performed in a holding step which occurs between two adjacent teleoperating steps, in which at the exit of a first teleoperating step the surgical instrument 20 is in a gripping or grip condition, for example on a surgical needle and/or on a biological tissue, such a grip must also be held during the subsequent holding step preparatory to the next teleoperating step (see in this regard the illustrations of FIGS. 8C and 8D).

According to an embodiment of the method, the aforesaid step (ii) comprising a loading and unloading cycle is performed only on a subset of tendons which are not involved in the actuation of the gripping degree of freedom.

Preferably, this option is implemented in combination with the aforementioned “hold squeeze” embodiment, which includes exiting teleoperation while the articulated end-effector 40 is gripping a needle or a tissue.

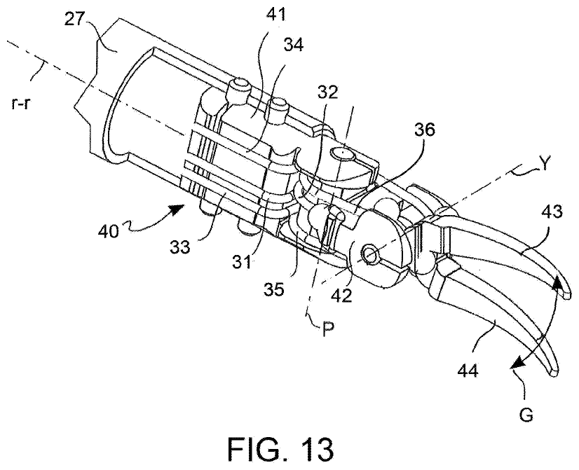

Typically, the gripping action affects four tendons (i.e., two pairs of antagonistic tendons, such as 33-34 and 35-36 shown in FIG. 13), but, according to possible variations, the affected tendons could be only two.

According to an implementation option, shown in FIG. 8C, the loading and unloading cycle is not performed, but simply the motorized actuators of the tendons involved in the grip are deactivated (“motor freeze”)—causing the decrease of the force, as shown in FIG. 8C—so that the tendons hold the grip on the gripped object.

According to another preferred implementation option, shown in FIG. 8D, the application of the gripping force is also held at the exit from the teleoperation in the gripping condition performed.

In accordance with an embodiment of the method, the robotic system 1 comprises control means 9 configured to control the motorized actuators 11, 12, 13, 14, 15, 16 to impart controlled movements and apply controlled forces to the tendons, preferably by means of the transmission elements 21, 22, 23, 24, 25, 26.

According to an implementation option of such an embodiment, in which a kinematic zero position of each of the motorized actuators 11, 12, 13, 14, 15, 16 is defined, and in which the method is applicable to a non-operating step between two teleoperation periods of the robotic system 1, the method comprises, at the beginning of a non-operating step, the following further steps:
storing as a known kinematic position of the end-effector device 40 of the surgical instrument 20 the position in which the end-effector device 40 is at the end of the previous teleoperating step, with respect to the kinematic zero position, to which a known kinematic position of each of the transmission elements POS_{kin-off} corresponds;
retracting the motorized actuators 11, 12, 13, 14, 15, 16 to remove, for each transmission element 21, 22, 23, 24, 25, 26, a respective position offset generated in the previous teleoperating step;
continuously applying, throughout the non-operating step of the surgical instrument, on each transmission element 21, 22, 23, 24, 25, 26, a respective recalibration force F, by means of a feedback control configured to keep the recalibration force F constant, so as to determine on each transmission element 21, 22, 23, 24, 25, 26 a respective position offset POS_{FC}(t) due to the application of the aforesaid respective recalibration force F.

In such a case, the method further comprises, at the end of the non-operating step, at the start of the next teleoperating step, the following further steps:
stopping the application of the recalibration force F to each transmission element 21, 22, 23, 24, 25, 26;
measuring and storing the position offset POS_{FC-off} determined on each transmission element 21, 22, 23, 24, 25, 26 at the end of the non-operating step, following the application of the recalibration force during the non-operating step just ended, and associating the position offsets POS_{FC-off} recorded for each transmission element 21, 22, 23, 24, 25, 26 to the aforesaid known kinematic position of the end device 40;
applying an operating and moving force as commanded by the control means 9, which are configured to determine the control force based on the operator's commands and taking into account the aforesaid stored position offsets POS_{FC-off} of each transmission element 21, 22, 23, 24, 25, 26.

According to an implementation option, the aforesaid recalibration force F corresponds to the holding force Fhold.

In accordance with an embodiment, the step of applying a recalibration force, on each transmission element, comprises applying a force to the transmission element by means of a feedback loop, in which the feedback signal corresponds to a force applied to a transmission element as actually detected by a respective force sensor which is operatively connected or connectable to the transmission element.

According to an implementation option, the aforesaid kinematic zero position comprises a fixed offset Prestretch_{off} resulting from a further step of pre-conditioning the surgical instrument, performed before using the surgical instrument.

In accordance with a specific implementation option, the aforesaid pre-conditioning step provides:
(i) locking at least one degree of freedom of the aforesaid at least one degree of freedom P, Y, G of the end-effector device 40;
(ii) tensile-stressing the respective at least one tendon, operatively connected to the at least one locked degree of freedom, by applying a conditioning force Fref, according to at least one time cycle, to the respective transmission element 21, 22, 23, 24, 25, 26 connected to the respective tendon to be tensile-stressed. The application of the conditioning force Fref is performed by a respective motorized actuator to stress the respective tendon.

Such at least one time cycle comprises at least one low-load period, in which a low conditioning force Flow is applied to the transmission element, which results in a respective low tensile load on the respective tendon; and at least one high-load period, in which a high conditioning force Fhigh is applied to the transmission element, which results in a respective high tensile load on the respective tendon.

The high conditioning force Fhigh can assume increasing value in two adjacent time cycles. In other words, a plurality of said time cycles is provided, in which, in at least two adjacent time cycles, the respective value of the high conditioning force Fhigh grows.

In the conditioning (pre-conditioning) step, a plurality of N time cycles can be provided, so as to determine an alternation between successive low-load periods Flow and high-load periods Fhigh, in which during the low-load periods of the n-th cycle a respective low conditioning force Flow_n is applied and in which during the high-load periods of the n-th cycle a respective high conditioning force Fhigh_n is applied.

According to an implementation, said low conditioning forces Flow_n of the different time cycles correspond to the same predetermined low conditioning force value Flow, and said high conditioning forces Fhigh_n correspond to gradually increasing high conditioning force values, until reaching a maximum high force value Fhigh_max.

According to an implementation, the high conditioning force value of the n-th time cycle is calculated according to the following formula:

$${F}_{high\_n} = \{ \begin{matrix}( \frac{{F}_{high\_max} - {F}_{low}}{N} ) n + {F}_{low} & n \leq N \\ {F}_{high\_max} & N < n \leq {N}_{c}\end{matrix} \}$$

where n is the current cycle, N is the total number of cycles, Nc is the number of cycles at constant Fhigh, and Fhigh_max is a settable value.

According to an implementation option, in said time cycle: (i) each of the at least one low-load period has a first time duration, and comprises a first holding sub-step having a first holding time duration during which a first force value corresponding to said low conditioning force Flow is applied; (ii) each of the at least one high-load period has a second time duration, and comprises a second holding sub-step having a second holding time duration during which a second force value corresponding to said high conditioning force Fhigh is applied. According to an implementation option, said first time duration comprises, in addition to the first holding sub-step with first holding time duration, a first ramp sub-step having a first ramp time duration, such that the sum of said first holding time duration and first ramp time duration corresponds to said first time duration; and said second time duration comprises, in addition to the second holding sub-step with second holding time duration, a second ramp sub-step having a second ramp time duration, such that the sum of said second holding time duration and second ramp time duration corresponds to said second time duration, and in which said first holding time duration is greater than said first ramp time duration and said second holding time duration is greater than said second ramp time duration. According to an embodiment, said first time duration is in the range of 0.2 seconds to 30.0 seconds, and said second time duration is in the range of 0.2 seconds to 5.0 seconds. Preferably, said first time duration is in the range of 1.0 seconds to 3.0 seconds, and said second time duration is in the range of 1.0 seconds to 3.0 seconds. According to an embodiment, said first ramp time duration is in the range of 0.2 to 10.0 seconds and said second ramp time duration is in the range of 0.2 to 2.0 seconds. According to an embodiment, said first holding time is in the range of 0.2 to 20.0 seconds and said second holding time is in the range of 0.2 to 3.0 seconds.

According to an implementation option, in the pre-conditioning step, said low conditioning force Flow has a value in the range of 0.2 N to 3.0 N, and said high conditioning force Fhigh has a value in the range of 8.0 N to 50.0 N. Preferably, said low conditioning force Flow has a value in the range of 1.0 N to 3.0 N, and said high conditioning force Fhigh has a value in the range of 10.0 N to 20.0 N.

The number N of time cycles of the pre-conditioning step is in the range of 1 to 30, and preferably, said number N of time cycles is in the range of 1 to 15, for example is less than 10, and/or more preferably, said number N of time cycles is in the range of 3 to 8.

As mentioned above, in the implementation options in which the transmission elements are not provided, the low and high conditioning forces Flow, Fhigh are applied to the tendons.

According to an embodiment of the method, the aforesaid step of retracting the motorized actuators comprises removing any position offset generated by further possible compensation steps of the transfer system.

According to an implementation option, the holding force Fhold and/or the recalibration force F are in the range of 0.1 to 5 N.

In accordance with an implementation option, said position offset must be less than a maximum allowable position offset dxA, for example in the range of 1 to 5 mm.

According to an embodiment, the method applies in cases in which the tendons are polymer tendons made of intertwined or braided polymer fibers.

According to an implementation option, the tendons are non-elastically deformable.

According to an embodiment, the method applies to a robotic system consisting of a robotic system for micro-surgical teleoperation, in which the surgical instrument is a micro-surgical instrument.

Referring again to FIGS. 1-13, further illustrations of the surgical instrument to which the method of the present invention is applied will be provided below, useful for an even better understanding of the method itself, as well as further details, by way of non-limiting example, on some embodiments of the method.

Some illustrative details about the aforementioned pre-conditioning step, or “pre-stretch,” are provided here.

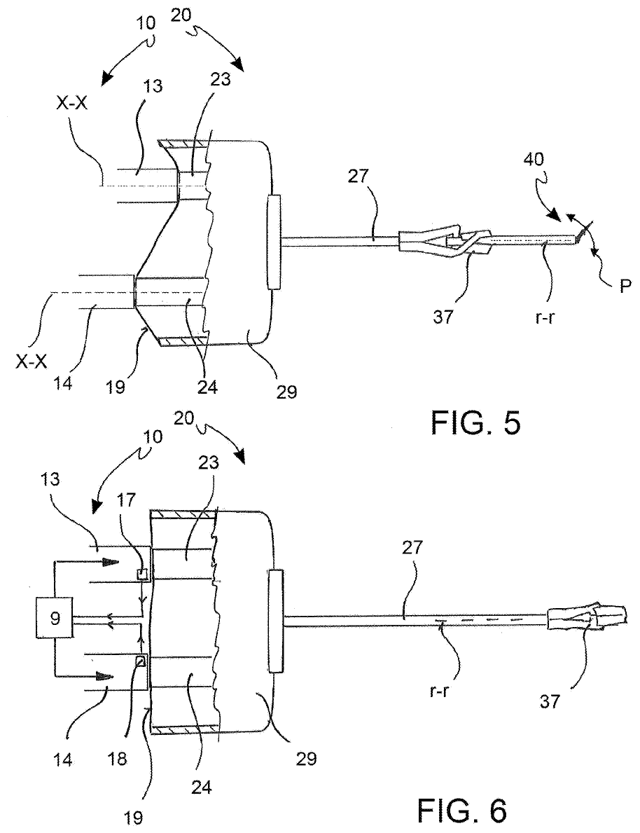

As diagrammatically shown, for example in the sequence of FIGS. 5 and 6, a constraining body 37 (shown here retractable along the shaft or rod 27 of the surgical instrument 20) can be fitted on the articulated end-effector device 40 to lock one or more degrees of freedom (in the example shown, the degree of freedom of pitch P is locked), so as to facilitate the execution of the conditioning procedure.

According to an implementation option, a constraining body 37 is provided for temporarily locking the articulating tip 40 in a predetermined configuration. The constraining body 37 can be retractable along the shaft 27 of the surgical instrument 20. The constraining body 37 can be a plug 37 or cap 37 which is not retractable along the shaft 27 of the surgical instrument 20, and for example can be removed distally with respect to the free end of the articulated end-effector device (end-effector) 40.

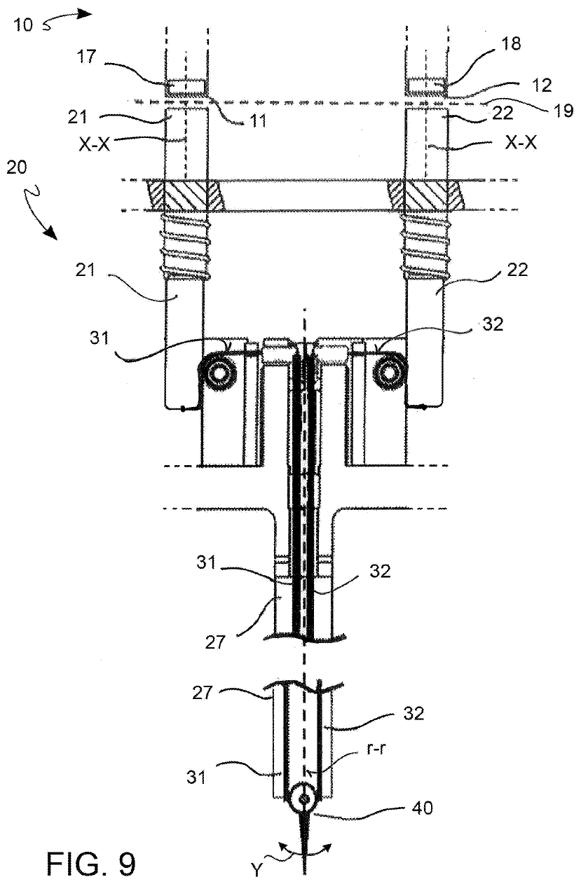

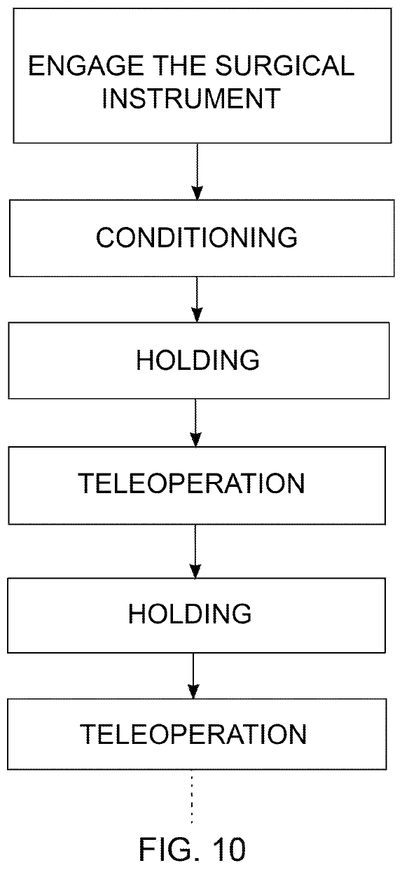

According to an implementation option, the at least one actuator 11, 12, 13, 14, 15, 16 is a linear actuator. In such a case, the at least one transmission element 21, 22, 23, 24, 25, 26 can be a linear transmission element, such as a piston adapted to move along a substantially straight path x-x, as shown for example in FIG. 9.

According to another implementation option, the at least one actuator is a rotary actuator, such as a winch. The at least one transmission element can be a rotary transmission element such as a cam and/or a pulley.

The articulated end-effector device 40 preferably comprises a plurality of links 41, 42, 43, 44 (e.g., rigid connection elements). At least some of such links, for example links 42, 43, 44 of FIG. 13, are connected to a pair of antagonistic tendons 31, 32; 33, 34; 35, 36.

As shown in the implementation example of FIG. 13, a pair of antagonistic tendons 31, 32 is mechanically connected to a link 42 to move such a link 42 with respect to a link 41 about a pitch axis P, in which the link 41 is shown integral with the shaft 27 of the surgical instrument 20; another pair of antagonistic tendons 33, 34 is mechanically connected to a link 43 (shown here having a free end) to move such a link 43 with respect to the link 42 about a yaw axis Y; yet another pair of antagonistic tendons 35, 36 is mechanically connected to a link 44 (shown here having a free end) to move such a link 44 with respect to the link 42 about a yaw axis Y; an appropriate joint activation of the links 43 and 44 about the yaw axis Y can result in an opening/closing or grip degree of freedom G. Those skilled in the art will appreciate that the configuration of the tendons and links, and of the degrees of freedom of the articulated end-effector 40, can vary with respect to those shown in FIG. 13 while remaining within the scope of the present disclosure.

According to an implementation option, three pairs of antagonistic tendons (31, 32), (33, 34), (35, 36) are provided to actuate three degrees of freedom (e.g., the degrees of freedom of pitch P, yaw Y, and grip G). In such a case, the surgical instrument 20 can comprise six transmission elements 21, 22, 23, 24, 25, 26 (for example six pistons, as shown for example in FIG. 4 where the tendons are diagrammatically shown in a dashed line), i.e., three pairs of antagonistic transmission elements (21, 22), (23, 24), (25, 26), intended for example to cooperate with three respective pairs of antagonistic motorized actuators (11, 12) (13, 14), (15, 16).

According to an implementation option, a sterile barrier 19 is interposed between at least the motorized actuators and the transmission elements, such as a sterile cloth made as a plastic sheet or other surgical sterile cloth material, such as fabric or non-woven fabric.

The joint inclusion of this sterile barrier 19 and of the sensors 17, 18 placed on the motorized actuators upstream of the sterile barrier 19 is particularly advantageous because it allows installing the active components of the control system (meaning here also the sensors) in a non-sterile environment, thus being able to reuse them for different interventions, avoiding assembling such components on the surgical instrument 20, which can be disposable and which works in a sterile environment downstream of the sterile barrier 19.

According to an implementation option, each polymer tendon of the at least one pair of antagonistic polymer tendons (31, 32), (33, 34), (35, 36) is preferably non-elastically deformable, although it can also be elastically deformable.

According to a preferred embodiment, each tendon of the at least one pair of antagonistic tendons of the surgical instrument 20 is made of polymer material.

Preferably, according to an implementation option, each tendon of the at least one pair of antagonistic tendons comprises a plurality of polymer fibers intertwined and/or braided to form a polymeric strand. According to an embodiment, each tendon of the at least one pair of antagonistic tendons comprises a plurality of high molecular weight polyethylene fibers (HMWPE, UHMWPE).

According to an implementation option, said at least one tendon can comprise a plurality of aramid fibers, and/or polyesters, and/or liquid crystal polymers (LCPs), and/or PBOs (Zylon®), and/or nylon, and/or high molecular weight polyethylene, and/or any combination of the foregoing.

According to an implementation option, each polymer tendon of the at least one pair of antagonistic polymer tendons is partially made of metal material and partially of polymer material, for example, formed by the intertwining of metal fibers and polymer fibers.

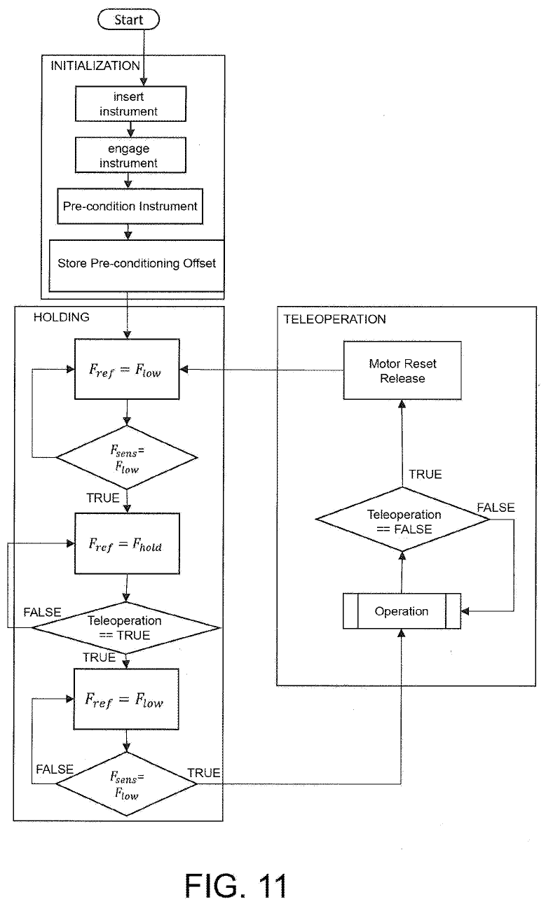

A particular embodiment of the method according to the invention, diagrammatically shown in the flow diagram of FIG. 11, is shown in more detail below, by way of non-limiting example.

In such a case, the method comprises the following steps reported in a preferred order of execution.

Firstly, an initialization step is provided, which comprises the following steps:
inserting the surgical instrument 20 into the appropriate connector or pocket 28 of the robotic manipulator 10;
engaging the surgical instrument 20, in which the motorized actuators 11, 12, 13, 14, 15, 16 of the robotic manipulator 10 move simultaneously to abut against each respective transmission element 21, 22, 23, 24, 25, 26 of the surgical instrument 20, avoiding moving the articulating tip 40 (i.e., the end-effector device 40) of the surgical instrument 20, and thus avoiding actuating the degrees of freedom P, Y of the articulating tip 40;
performing a step of pre-stretching the tendons 31, 32, 33, 34, 35, 36;
optionally, storing the offset position (Prestretch_{off}) of the motorized actuators 11, 12, 13, 14, 15, 16 at the end of the pre-stretching step. The storage of this parameter Prestretch_{off} occurs preferably when the surgical instrument 20 (i.e., the degrees of freedom of the articulating tip 40 of the surgical instrument 20), is at the kinematic zero, and this allows having a constant reference of the initial position before the first teleoperation. Due to subsequent position corrections of the motorized actuators 11, 12, 13, 14, 15, 16, such a position can be used to trace a kinematic coherence position between the motorized actuators and degrees of freedom P, Y, G of the surgical instrument 20.

After the aforesaid initialization step, the method provided a teleoperation preparation step, comprising the application of a first holding step.

The first holding step comprises the following actions:
a feedback force control is used on the six motorized actuators 11, 12, 13, 14, 15, 16, independently, i.e., individually on each motorized actuator, in order to hold the position of the motorized actuators and the transmission elements 21, 22, 23, 24, 25, 26 abutting therewith, reached during the pre-stretching step and/or during the engagement step;
the motorized actuators apply an applied force Fref equal to a minimum force value Flow;
if the detected force Fsens, by means of force sensors 17, 18, corresponds to the minimum force value Flow, then the motorized actuators apply an applied force Fref equal to a holding force value Fhold greater than the minimum force value Flow to maintain tension on the respective tendons and avoid their relaxation; the holding force value Fhold is preferably determined experimentally and can vary depending on the type of surgical instrument used; such a holding force value Fhold is determined so as to allow, after a first pre-stretching procedure, holding the elongation as constant as possible, i.e., in order to prevent the tendons from undergoing a shortening due to the recovery of the elongation deformation of the tendons previously subjected to stress, while preventing the tendons from undergoing a further elongation due to the phenomenon of reconfiguration of the structure of the tendons;
at this point the system verifies that the operator has indicated the will to enter teleoperation (“operation==TRUE”), for example by pressing a control pedal;
the motorized actuators again apply said minimum force value Flow; the re-application of the minimum-level force allows the force to be discharged to the motion transmission joints inside the surgical instrument; this allows the friction generated by the tendons-joints coupling to be reduced during teleoperation, and in turn the decrease in friction reduces the non-matching effects between the master and slave devices of the robotic system, during teleoperation;
if the force Fsens detected by force sensors 17, 18 corresponds to the minimum force value Flow, then the system enables entry into a first teleoperating step;
the offset positions of the motorized actuators 11, 12, 13, 14, 15, 16 are stored at the end of the pre-stretching step (Prestretch_{off}).

After the aforesaid first holding step, the method provides performing a first teleoperating step, in which:
the entry and/or entry enablement during the teleoperating step is subject to a teleoperation request command (“operation==TRUE”), such as pressing a control pedal by the operator;
the teleoperating step comprises the enslavement (i.e., following) of the motorized actuators to a respective master device 3, in which the motorized actuators can be moved according to kinematic laws and in which the force control can be disabled.

Subsequently, the first teleoperating step is interrupted and the method provides the system performing a second teleoperation preparation step in which a second holding step is applied.

The second holding step comprises the following actions:
a feedback force control is used on the six motorized actuators 11, 12, 13, 14, 15, 16, independently, in order to balance the forces applied on each transmission element 21, 22, 23, 24, 25, 26 following the change in configuration of the position of the motorized actuators with respect to the respective stored offset position at the end of the pre-stretching step, according to the relationship:

$${M}_{p o s} ( t ) = {Pres tretch}_{off} + P o {s}_{Kinoff} + P o {s}_{F C} ( t )$$

where:
Mₚₒₛ(t) is the position of each of the motorized actuators with respect to a motor reference system, for example positioned at the distal end of each motorized actuator;
Prestretch_{off} is the stored offset after completion of the pre-stretching procedure with respect to the above motor reference system;
POSK_{inoff} is the offset generated by the kinematic laws stored at the exit of the aforesaid first teleoperating step;
POS_{FC}(t) is the displacement of the motorized actuators generated by the force control as a function of time.

During this second holding step, the position offset of the motorized actuators with respect to the respective offset position stored at the end of the pre-stretching step is stored, i.e.:

$${M}_{p o s} ( t ) = {Prestretch}_{off} + Po {s}_{FCoff} + P o {s}_{K i n} ( t )$$

where:
Pos_{Kin}(t) is the displacement of the motorized actuators generated by the kinematic control.

Therefore, the position offset of the motorized actuators, stored after the second holding step, is expressed by the formula:

$$P o {s}_{FCoff} = {M}_{p o s} ( {T}_{teleop ON} ) - {Prestretch}_{off} - {Pos}_{Kinoff}$$

As described above with reference to the first holding step, during the second holding step the following actions are carried out:
the motorized actuators apply an applied force Fref equal to a minimum force value Flow;
if the force Fsens detected by force sensors 17, 18 corresponds to the minimum force value Flow, then the motorized actuators apply an applied force Fref equal to a holding force value Fhold greater than the minimum force value Flow to maintain tension on the respective tendons and avoid the relaxation thereof;
at this point the system verifies that the operator has indicated the will to enter teleoperation (“operation==TRUE”), for example by pressing a control pedal;
the motorized actuators again apply the aforesaid minimum force value Flow;
if the force Fsens detected by force sensors 17, 18 corresponds to the minimum force value Flow, then the system enables entry into a first teleoperating step.

After the aforesaid second holding step, a second teleoperating step is performed, which can be substantially similar to the first teleoperating step.

Entering a teleoperating step after performing a holding procedure makes the surgical instrument 20 ready to move in any direction, reducing the “lost motion” which can have been generated by the lockage of the surgical instrument in a configuration in which the motorized actuators insist on the transmission elements with different forces.

The alternation between preparation steps, each comprising the aforesaid holding step, and teleoperating steps, can continue in a determined or undetermined manner.

At the end of a teleoperating step, but also at the end of each teleoperating step, and before a holding step, a release step (“release motor offset”) can be included, in which this release step is entered by means of a command to exit the teleoperation (“Operation==FALSE”), for example the release of a control pedal, applied by the user in which the possibility of teleoperating the surgical instrument 20 is disabled.

In this release step, the stored motorized actuator position offset (POS_{FCoff}) is removed. This release step allows resetting any positioning errors previously accrued in a holding step, thus allows deleting a possible position drifting, i.e.:

$${M}_{p o s} ( t ) = {Prestretch}_{off} + {Pos}_{Kinoff}$$

According to an implementation option, the minimum force value, Flow, is a minimum force value with which the motorized actuators come into contact (i.e., abut) with the transmission elements.

According to an implementation option, the holding force value Fhold is a force value greater than the minimum force value Flow and is used to maintain tension on the respective tendons 31, 32, 33, 34, 35, 36 and prevent the relaxation thereof.

According to several possible implementation options of the method, the aforesaid value Fhold can be predetermined, i.e., calculated by experimental tests on the particular type of tendon used.

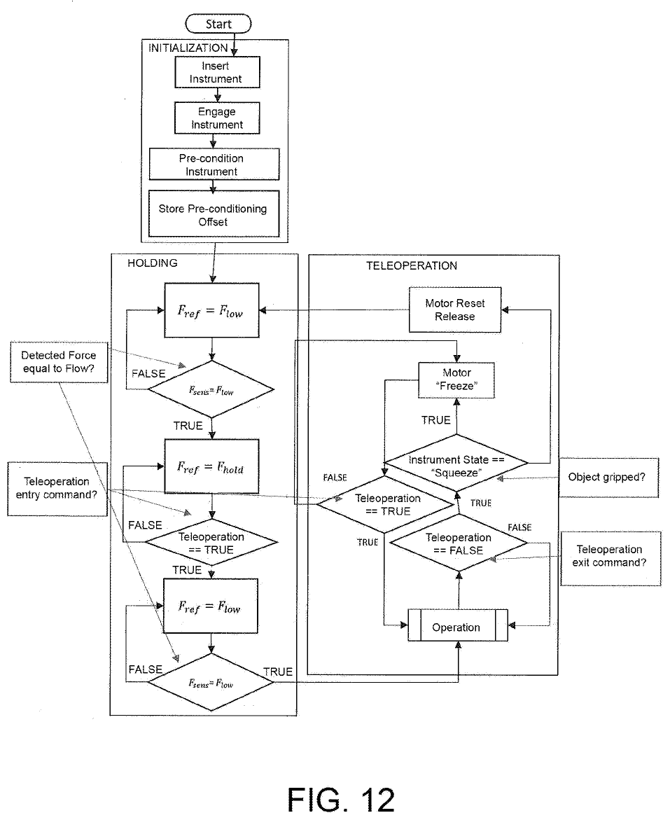

The two force values Flow and Fhold can be alternated so as to avoid a possible undesired displacement of the end-effector device 40 during the holding step. For example, these force values Flow and Fhold are alternated as shown for example in the diagrams shown in FIGS. 11 and 12.

According to another embodiment of the method (already described above), any of the holding steps, or even all of the holding steps, use an in-position control, in place of the feedback force control.

According to an implementation option of the method, at the exit of a teleoperating step in which there is an intensive actuation of the degree of freedom of grip (grip, G), the system performs a holding step taking into account such an intensive actuation of the degree of freedom of grip so as to ensure holding the kinematic matching, compensating for the elongation of the tendons due to the application of the relatively very high grip force for a relatively long time. Thereby, it is possible to avoid a possible kinematic imbalance caused by the fact that only some tendons (for example, a subset of two-four tendons out of six) have been stressed more than the other tendons and therefore may have been subject to elongation to a greater extent than the other tendons.

As shown for example in FIG. 13, the degree of freedom of grip (G) is activated by the action exerted by two pairs of antagonistic tendons (33, 34) and (35, 36) to hold the grip on a body 45 which can be for example a biological tissue or a surgical needle.

As described above, the holding step does not necessarily comprise loading and unloading cycles, but can only comprise application of a loaded state (force Fhold).

According to an implementation option of the method, in which the surgical instrument 20 exits a teleoperating step in a gripping condition (active degree of freedom of grip G, which state is also referred to as “squeeze”), the actuation tendons of such a degree of freedom of grip are tensile-stressed. In this case, the holding step comprises applying a loaded state in which the holding force is at least equal to the gripping force. Thereby, losing the gripping condition is avoided.

According to an implementation option, the holding force corresponds to the gripping force.

According to an implementation option, the system recognizes the aforesaid condition of exiting a teleoperating step in a gripping condition (active degree of freedom of grip G) if the master device of the teleoperated system identifies a “squeeze” state.

According to an implementation option, the system recognizes the aforesaid condition of exit from a teleoperating step in a gripping condition (active degree of freedom of grip G) if the force measured on the motorized actuators and/or on the transmission means operatively associated with the actuation tendons of the degree of freedom of grip is greater than a predefined threshold value.

According to an implementation option, the holding force can be at least equal (e.g., corresponding) to the gripping force only on the actuation tendons of the degree of freedom of grip. Therefore, if the actuation tendons of the degree of freedom of grip are a pair of antagonistic tendons, then the system applies a loaded state comprising applying a holding force, avoiding applying a loading and unloading cycle, on such a pair of antagonistic tendons.

If, on the other hand, the actuation tendons of the degree of freedom of grip are two pairs of antagonistic tendons, then the system applies a loaded state comprising the application of a holding force Fhold, avoiding the application of a loading and unloading cycle, on such two pairs of antagonistic tendons.

Alternatively, the holding force can be at least equal (e.g., corresponding) to the gripping force on all the tendons of the surgical instrument 20.

According to a different implementation, in which the surgical instrument 20 exits a teleoperating step in a gripping condition (active degree of freedom of grip G, “squeeze” state), and thus the actuation tendons of such a degree of freedom of grip are tensile-stressed, the robot avoids carrying out the holding procedure/step (“motor freeze” in FIG. 12) on a subset of tendons comprising the aforesaid actuation tendons of the degree of freedom of grip. In this case, the holding step comprises applying a loading and unloading cycle as previously described.

If the actuation tendons of the degree of freedom of grip are a pair of antagonistic tendons, then the holding step on such a pair of antagonistic tendons is avoided.

If, on the other hand, the actuation tendons of the degree of freedom of grip are two pairs of antagonistic tendons, as shown in the example of FIG. 13, then the holding step on such two pairs of antagonistic tendons is avoided, while the holding step is performed on the other tendons (the tendons 31 and 32 of FIG. 13).

Preferably, the system is adapted to store this occurred exit from a teleoperating step in a gripping condition (active degree of freedom of grip G), in order to subsequently compensate (for example at the next exit from a teleoperating step) the failure to carry out the holding step on the actuation tendons of the degree of freedom of grip, carrying out a holding step.

Referring again to FIGS. 1-13, a teleoperated robotic surgery system 1 is described below comprising a plurality of motorized actuators 11, 12, 13, 14, 15, 16, at least one surgical instrument 20 and control means 9.

The aforesaid at least one surgical instrument 20 comprises an articulated end-effector device 40 having at least one degree of freedom P, Y, G; and at least one pair of antagonistic tendons 31, 32; 33, 34; 35, 36, mounted in the surgical instrument 20 so as to be operably connectable to both respective motorized actuators, and to respective links of the end device 40 to actuate at least one degree of freedom associated therewith (between the aforesaid at least one degree of freedom P, Y, G), thus determining antagonistic effects.

The control means 9 of the system 1 are configured to control the execution of the following actions:
(i) establishing a univocal correlation between a set of movements of the motorized actuators 11, 12, 13, 14, 15, 16 of the robotic system 1 and a respective movement of the articulated end-effector device 40 of the surgical instrument 20;
(ii) performing a holding step comprising:
   stressing, through tensile-stressing, at least one pair of antagonistic tendons 31, 32; 33, 34; 35, 36 and keeping the tendons in a tensile-stressed state, by applying a holding force Fhold to the tendons, said holding force Fhold being adapted to determine a loaded state of the tendons;
   enabling the entry of the surgical instrument 20 in a teleoperation state, upon receiving a command indicating a will to enter teleoperation.

According to various possible embodiments of the system 1, the control means are configured to control the robotic system so as to perform a method of teleoperation preparation according to any of the previously illustrated embodiments of such a method.

As can be seen, the objects of the present invention as previously indicated are fully achieved by the method described above by virtue of the features disclosed above in detail, and as already disclosed above in the summary of the invention.

In order to meet contingent needs, those skilled in the art may make changes and adaptations to the embodiments of the method described above or can replace elements with others which are functionally equivalent, without departing from the scope of the following claims. All the features described above as belonging to a possible embodiment can be implemented irrespective of the other embodiments described.

### LIST OF NUMERICAL REFERENCES

| | |
|---|---|
| 1 | Robotic system for teleoperated surgery |
| 2 | Slave assembly of the robotic system |
| 3 | Master console |
| 9 | Controller, i.e., control unit |
| 10 | Robotic system manipulator |
| 11, 12, 13, 14, 15, 16 | Motorized actuators of the manipulator |
| 17, 18 | Force sensors, or load cells |
| 19 | Sterile barrier |
| 20 | Surgical instrument |
| 21, 22, 23, 24, 25, 26 | Surgical instrument transmission elements |
| 27 | Shaft |
| 28 | Pocket |
| 29 | Surgical instrument backend, or transmission |
| | interface portion |
| 31, 32, 33, 34, 35, 36 | Tendons |
| 37 | Constraining body, or plug, or cap |
| 40 | End effector device, or articulating tip, or end- |
| | effector, of the surgical instrument |
| 41, 42, 43, 44 | Links of the articulating tip |
| 45 | Body |
| x-x | Straight direction |
| r-r | Centerline |
| P, Y, G | Degree of freedom, pitch, yaw, grip, respectively, |
| | of the end-effector device |
| Fref | Applied force |
| Fsens | Force detected by force sensors |
| Flow | Low force value |
| Fhigh | High force value |

1. A method of teleoperation preparation in a teleoperated robotic surgery system, to be performed during a non-operating step, in which the system is not performing a teleoperation,
wherein the robotic system comprises a plurality of motorized actuators and at least one surgical instrument,
wherein the at least one surgical instrument comprises:
an articulated end-effector having at least one degree of freedom;
at least one pair of antagonistic tendons, mounted in said surgical instrument to be operatively connectable to both respective motorized actuators and respective links of the end-effector to actuate at least one degree of freedom associated therewith, among said at least one degree of freedom, thus determining antagonistic effects;
wherein the method comprises the steps of:
(i) establishing a univocal correlation between a set of movements of the motorized actuators of the robotic system and a respective movement of the articulated end-effector of the surgical instrument;
(ii) performing a holding step comprising:
stressing, through tensile-stressing, at least one pair of antagonistic tendons and keeping the tendons in a tensile-stressed state, by applying a holding force to the tendons, said holding force being adapted to determine a loaded state of the tendons,
providing a command indicating a will to enter teleoperation;
enabling the surgical instrument to enter a teleoperation state.

2. The method according to claim 1, comprising, after steps (i)-(ii), the step of:
(iii) teleoperating by the surgical instrument of the robotic system, wherein the holding step (ii) and the teleoperating (iii) step are repeated, so that a holding step (ii) is performed between two adjacent teleoperating steps (iii).

3. (canceled)

4. The method according to claim 1, wherein the surgical instrument further comprises:
a plurality of transmission elements, each operatively connectable to a respective at least one motorized actuator;
wherein said step of stressing is performed by the transmission elements, operated and controlled by the respective motorized actuators;
and wherein said transmission elements are rigid.

5. The method according to claim 1, wherein a kinematic zero position of each of the motorized actuators is defined, and wherein the method comprises, during the holding step (ii) after said step of stressing at least one pair of antagonistic tendons, the further step of:
storing a possible position offset of each of the motorized actuators with respect to the respective stored kinematic zero position.

6. The method according to claim 1, wherein, during the holding step (ii), the step of stressing at least one pair of antagonistic tendons comprises at least one loading and unloading cycle, wherein each loading and unloading cycle includes applying a high force to determine a loaded state of the pair of tendons and applying a low force to determine an unloaded state of the pair of tendons,
wherein said high force corresponds to said holding force, and said low force is a lower force than said holding force.

7. The method according to claim 6, wherein, in each of said loading and unloading cycles, first the low force is applied and then the high or holding force is applied.

8. The method according to claim 6, wherein, in said holding step (ii), between the step of providing a command indicating the will to enter teleoperation and the step of enabling the entry into a teleoperation state, the method comprises the further step of:
applying said low force to the tendons, to have the tendons under tensile load according to said unloaded state of the loading and unloading cycle.

9. The method according to claim 6, comprising the further steps of:
detecting the forces applied to all the tendons at an exit of a teleoperating step;
identifying a minimum force among said detected forces;
bringing all the tendons to an intermediate tensile stress condition corresponding to said minimum force.

10. The method according to claim 8, wherein said step of bringing all the tendons to an intermediate stress condition corresponding to the minimum force is performed following specific and/or different loading and/or unloading curves for each tendon, as a function of a starting force value detected for each tendon.

11. The method according to claim 9, wherein said step of applying the holding force to the tendons comprises:
bringing all the tendons to an intermediate stress condition corresponding to said minimum force value, each tendon according to a respective specific load curve dependent on a respective detected starting force value, so that the load is equally distributed between the antagonistic tendons of one or more pairs of antagonistic tendons;
then bringing all the tendons to a loaded stress condition, corresponding to said holding force.

12. The method according to claim 1, wherein the teleoperating step begins with a predeterminable teleoperation start force applied to the tendons which is lower than said high holding force value.

13. The method according to claim 1, wherein said step of stressing the tendons comprises measuring or detecting the force acting on the tendons during the loading cycle, and reaching the holding force value, by the motorized actuators, through a feedback force control procedure based on the actual force acting on the tendons as detected or measured, or
wherein said step of stressing the tendons comprises measuring or detecting a position offset of the motorized actuators with respect to respective initial values, predetermined or stored at an end of the previous teleoperating step, and performing the loading cycle, by the motorized actuators, through a feedback position control procedure based on said position offsets as detected or measured or stored.

14-16. (canceled)

17. The method according to claim 1, wherein, during the holding step (ii), the at least one pair of tendons is stressed by a loaded state corresponding to a gripping action of the end-effector of the surgical instrument, so that during the holding step the surgical instrument is in a gripping condition, or
wherein said holding step (ii) comprising a loading and unloading cycle is performed only on a sub-set of tendons which are not involved in actuation of the gripping degree of freedom.

18. (canceled)

19. The method according to claim 1, wherein the robotic system comprises a controller configured to control the motorized actuators to impart controlled movements and apply controlled forces to the tendons, by transmission elements operatively connected to respective tendons,
wherein a kinematic zero position of each of the motorized actuators is defined, the method being applicable to a non-operating step between two teleoperation periods of the robotic system,
wherein the method comprises, at the beginning of a non-operating step, the following further steps:
storing as a known kinematic position of the end-effector of the surgical instrument the position in which the end-effector is at an end of the previous teleoperating step, with respect to the kinematic zero position, to which a known kinematic position of each of the transmission elements corresponds;
retracting the motorized actuators to remove, for each transmission element, a respective position offset generated in the previous teleoperating step;
continuously applying, throughout the non-operating step of the surgical instrument, on each transmission element, a respective recalibration force, by a feedback control configured to keep the recalibration force constant, to determine on each transmission element a respective position offset due to application of the respective recalibration force; and
wherein the method further comprises, at the end of the non-operating step, at the start of the next teleoperating step:
stopping the application of the recalibration force to each transmission element;
measuring and storing the position offset determined on each transmission element at the end of the non-operating step, following the application of the recalibration force during the non-operating step just ended, and associating the position offsets recorded for each transmission element to said known kinematic position of the end-effector;
applying an operating and moving force as commanded by the controller, wherein the controller is configured to determine the control force based on the operator's commands and taking into account said stored position offsets of each transmission element.

20. (canceled)

21. The method according to claim 19, wherein said recalibration force corresponds to the holding force, or
wherein the step of applying a recalibration force, on each transmission element, comprises applying a force to the transmission element by a feedback loop, wherein the feedback signal corresponds to a force applied to a transmission element as actually detected by a respective force sensor which is operatively connectable to the transmission element.

22. (canceled)

23. The method according to claim 19, wherein said kinematic zero position comprises a fixed offset resulting from a further step of pre-conditioning the surgical instrument, carried out before using the surgical instrument.

24. The method according to claim 1, further comprising a pre-conditioning step comprising:
(i) locking at least one degree of freedom of said at least one degree of freedom (P, Y, G) of the end-effector;
(ii) tensile-stressing the respective at least one tendon, operatively connected to said at least one locked degree of freedom, by applying a conditioning force, according to at least one time cycle, to the respective transmission element connected to said respective at least one tendon to be tensile-stressed;
wherein said at least one time cycle comprises:
at least one low-load period, in which a low conditioning force is applied to said respective transmission element, which results in a respective low tensile load on the respective tendon;
at least one high-load period, in which a high conditioning force is applied to said respective transmission element, which results in a respective high tensile load on the respective tendon.

25. The method according to claim 24, wherein a plurality of said time cycles is provided, and wherein, in at least two adjacent time cycles, the respective value of the high conditioning force increases, and/or
wherein a plurality of N time cycles is provided, to determine an alternation between successive low-load periods and high-load periods, wherein during the low-load periods of the n-th cycle a respective low conditioning force is applied, and wherein during the high-load periods of the n-th cycle a respective high conditioning force is applied,
wherein said low conditioning forces of the different time cycles correspond to a same predetermined low conditioning force value, and wherein said high conditioning forces correspond to gradually increasing high conditioning force values, until reaching a maximum high force value.

26. (canceled)

27. The method according to claim 21, wherein said step of retracting the motorized actuators comprises removing any position offset generated by further elastic or plastic compensation steps of the transfer system.

28. The method according to claim 1, wherein the holding force and/or the recalibration force is in the range of 0.1-5 N, or
wherein said position offset must be less than a maximum allowable position offset,
wherein said maximum allowable offset is in the range of 1-5 mm.

29. (canceled)

30. A teleoperated robotic surgery system comprising a plurality of motorized actuators, at least one surgical instrument and a controller,
wherein the at least one surgical instrument comprises:
an articulated end-effector having at least one degree of freedom;
at least one pair of antagonistic tendons, mounted in said surgical instrument to be operatively connectable to both respective motorized actuators and respective links of the end-effector to actuate at least one degree of freedom associated therewith, among said at least one degree of freedom, thus determining antagonistic effects;
wherein the controller is configured to control execution of the following actions:
(i) establishing a univocal correlation between a set of movements of the motorized actuators of the robotic system and a respective movement of the articulated end-effector of the surgical instrument;
(ii) performing a holding step comprising:
stressing, through tensile-stressing, at least one pair of antagonistic tendons and keeping the tendons in a tensile-stressed state, by applying a holding force to the tendons, said holding force being adapted to determine a loaded state of the tendons;
enabling entry of the surgical instrument in a teleoperation state, upon receiving a command indicating a will to enter teleoperation.

31-49. (canceled)

50. The method according to claim 6, wherein said step of stressing the tendons comprises measuring or detecting the force acting on the tendons during the unloading cycle, and reaching the low force value, by the motorized actuators, through a feedback force control procedure based on the actual force acting on the tendons as detected or measured; or
wherein said step of stressing the tendons comprises measuring or detecting a position offset of the motorized actuators with respect to respective initial values, predetermined or stored at the end of the previous teleoperating step, and performing the unloading cycle, by the motorized actuators, through a feedback position control procedure based on said position offsets as detected or measured or stored.

51. The method according to claim 1, wherein the teleoperating step begins with a predeterminable teleoperation start force applied to the tendons which is lower than said high holding force value,
wherein said predeterminable teleoperation start force is substantially equal to the low holding force, and
wherein a transition between the high holding force and the teleoperation start force is controlled by the user by activating a control pedal.

52. The method according to claim 6, comprising the further steps of:
detecting the forces applied to all the tendons at an exit of a teleoperating step;
identifying the minimum force among said detected forces;
bringing all the tendons to an intermediate tensile stress condition corresponding to said minimum force value;
then bringing all the tendons to an unloaded stress condition, corresponding to said low force; and/or
then bringing all the tendons to a loaded stress condition, corresponding to said high holding force.
